(19) United States
(12) Patent Application Publication
(10) Pub. No.: US 2007/0270371 A1
(43) Pub. Date: Nov. 22, 2007

(54) Dosing and scheduling of oligomers

(76) Inventor: Bob D. Brown, Millington, NJ (US)

Correspondence Address: KENYON & KENYON LLP, ONE BROADWAY, NEW YORK, NY, 10004, US

(21) Appl. No.: 11/731,446

(22) Filed: Mar. 30, 2007

Related U.S. Application Data
(60) Provisional application No. 60/787,846, filed on Mar. 31, 2006.

(51) Int. Cl. A61K 31/70; A61P 43/00

(52) U.S. Cl. 514/44

(57) ABSTRACT

The present invention relates to a method for treating a human patient having a proliferative disorder by administering an effective amount of a pre-treatment to the patient prior to administering an effective amount of a nucleotide-based composition that inhibits the over-expression of a target gene that causes the proliferative disorder. The present invention includes pre-treatment with one or more radiotherapy such as an X ray, a proton beam, an electron beam; hyperthermic therapy, ultrasonic therapy, chemotherapy and a biologic therapy. The invention also relates to an improved dose and scheduling sequence for treating a human patient in need thereof with such a nucleotide-based inhibitor of gene expression.

This application claims priority to Application Ser. No. 60/787,846 filed on Mar. 31, 2006, and is incorporated by reference herein in its entirety.

### FIELD OF THE INVENTION

The present invention relates to a method for treating a human patient having a proliferative disorder such as cancer or an autoimmune disease caused by, or made more difficult to treat by, the over-expression of a target gene, including but not limited to an oncogene including a cancer promoting gene or a drug resistance gene, by administering an effective amount of a pre-treatment to the patient prior to administering an effective amount of a nucleotide-based composition that inhibits the expression of such a gene. More particularly the pre-treatment is one or more of radiotherapy such as an X ray, a proton beam, an electron beam; hyperthermic therapy, ultrasonic therapy, chemotherapy and a biologic therapy. The invention also relates to an improved dose and scheduling sequence for treating a human patient in need thereof with such a nucleotide-based inhibitor of gene expression.

### BACKGROUND

Previous approaches to the treatment of proliferative disorders including cancer suffer from a lack of specificity. Most drugs that have been developed are natural products or derivatives that block metabolic pathways (Araujo, et al., 2006, Curr. Cancer Drug Targets. 6:77-87) or randomly interact with DNA. Moreover, most cancer treatment are accompanied by serious dose-limiting toxicities due to low therapeutic indices. For example, the majority of anti-cancer drugs when administered to a patient kill not only cancer cells but also normal, non-cancerous cells. Because of these deleterious effects, treatments that more specifically affect cancerous cells are needed.

Advances in the understanding of the molecular mechanisms in tumor progression have identified many potential gene targets, including oncogenes, drug resistance genes, and growth and cell cycle regulating genes, involved in the transformation of cells, and in the maintenance of a cancerous state. Notably, disrupting the transcription of these genes, or otherwise inhibiting the effects of their protein products, can have a favorable therapeutic result. The role of oncogenes in the etiology of many human cancers has been reviewed in Bishop, 1987, “Cellular Oncogenes and Retroviruses,” Science, 235:305-311. For example, in many types of human cancers, a gene known as Bcl-2 (B cell lymphoma/leukemia-2) is over-expressed, and this over-expression may be associated with tumorigenicity (Tsujimoto et al., 1985, “Involvement of the Bcl-2 gene in human follicular lymphoma”, Science 228: 1440-1443). The Bcl-2 gene is thought to contribute to the pathogenesis of cancer, as well as to resistance to treatment, primarily by prolonging cell survival rather than by accelerating cell division.

The human Bcl-2 gene is implicated in the etiology of certain leukemias, lymphoid tumors, lymphomas, neuroblastomas, and nasopharyngeal, prostate, breast, and colon carcinomas. (Croce et al., 1987, “Molecular Basis of Human B and T Cell Neoplasia,” in: Advance in Viral Oncology, 7:35-5 1, G. Klein (ed.), New York: Raven Press; Reed et al., 1991; “Differential expression of Bcl-2 proto-oncogene in neuroblastoma and other human tumor cell lines of neural origin”, Cancer Res. 51:6529-38; Yunis et al., 1989, “Bcl-2 and other genomic alterations in the prognosis of large-cell lymphomas”, N. Engl. J. Med. 320: 1047-54; Campos et al., 1993, “High expression of Bcl-2 protein in acute myeloid leukemia is associated with poor response to chemotherapy”, Blood 81 :3091-6; McDonnell et al., 1992, “Expression of the proto-oncogene Bcl-2 and its association with emergence of androgen-independent prostate cancer”, Cancer Res. 52:6940-4; Lu et al., 1993, “Bcl-2 proto-oncogene expression in Epstein Barr Virus-Associated Nasopharyngeal Carcinoma”, Int. J. Cancer 53:29-35; Bonner et al., 1993, “Bcl-2 proto-oncogene and the gastrointestinal mucosal epithelial tumor progression model as related to proposed morphologic and molecular sequences”, Lab. Invest. 68:43A). Bcl-2 has been found to be over-expressed in a variety of tumors including non-Hodgkin's lymphoma, lung cancer, breast cancer, colorectal cancer, prostate cancer, renal cancer and acute and chronic leukemias (Reed, 1995, “Regulation of apoptosis by Bcl-2 family proteins and its role in cancer and chemoresistance”, Curr. Opin. Oncol. 7:541-6).

Antisense oligonucleotides provide potential therapeutic tools for specific disruption of oncogene and other target gene function. These oligomers have a sequence complementary or partially complementary to DNA or pre-mRNA or mRNA regions of a target gene, and form a duplex by hydrogen-bonded base pairs. This hybridization can disrupt expression of both the target mRNA and the protein which it encodes, and thus can interfere with downstream interactions and signaling. Since one mRNA molecule gives rise to multiple protein copies, inhibition of pre-mRNA or mRNA can be more efficient and more specific than causing disruption at the protein level, e.g., by inhibition of an enzyme's active site or other protein structure based function.

Oligonucleotides complementary to mRNA of the c-myc oncogene have been used to specifically inhibit production of c-myc protein, thereby arresting the growth of human leukemic cells in vitro (Holt et al., 1988, Mol. Cell Biol. 8:963-73; Wickstrom et al., 1988, Proc. Natl. Acad. Sci. USA, 85:1028-32). Oligonucleotides have also been employed as specific inhibitors of retroviruses, including the human immunodeficiency virus (Zamecnik and Stephenson, 1978, Proc. Natl. Acad. Sci. USA, 75:280-4; Zamecnik et al., 1986, Proc. Natl. Acad. Sci. USA, 83:4143-6).

The use of antisense oligonucleotides, with their ability to target and inhibit individual cancer-related genes, has shown promise in preclinical cancer models and in clinical trials. Phosphorothioate antisense oligonucleotides have shown an ability to inhibit Bcl-2 expression in vitro and to eradicate tumors in mouse models with lymphoma xenografts. Resistance to chemotherapy of some cancers has been linked to expression of the Bcl-2 oncogene (Grover et al., 1996, “Bcl-2 expression in malignant melanoma and its prognostic significance”, Eur. J. Surg. Oncol. 22(4):347-9). Administration of a Bcl-2 targeting oligonucleotide can selectively reduce Bcl-2 protein levels in tumor xenografts in laboratory mice (Jansen et al., 1998, “Bcl-2 antisense therapy chemosensitizes human melanoma in SCID mice”, Nat. Med. 4(2):232-4). Moreover, administration of a Bcl-2 antisense oligonucleotide can make tumor xenografts in laboratory mice more susceptible to chemotherapeutic agents (Jansen et al., 1998, “Bcl-2 antisense therapy chemosensitizes human melanoma in SCID mice”, Nat. Med. 4(2):232-4). In mice, systemic treatment with a Bcl-2 antisense oligonucleotide reduced Bcl-2 protein and enhanced apoptosis. Treatment with Bcl-2 antisense oligonucleotide alone had modest antitumor activity while enhanced antitumor activity was observed when combined with other conventional therapies such as chemotherapeutic agents. Clinical trials in a number of tumor types utilizing a Bcl-2 antisense oligonucleotide also have demonstrated activity. However, commonly these responses subsequently give rise to clonogenic survivals and disease progression as a result of resistance to these treatment modalities. Thus, there remains a compelling need to extend these antitumor treatments to combat cancer in humans.

The prognosis of many cancer patients is poor despite the increasing availability of biologic, drug, and combination therapies. For example, although dacarbazine (DTIC) is commonly used to treat metastatic melanoma, few patients have demonstrated long-term improvement. In fact, an extensive phase III clinical trial did not demonstrate any better survival when DTIC was used in combination with other chemotherapeutic agents such as cisplatin, carmustine, and tamoxifen (Chapman et al., 1999, “Phase III multicenter randomized trial of the Dartmouth regimen versus dacarbazine in patients with metastatic melanoma”, J. Clin. Oncol. 17(9):2745-5 1). Furthermore, although many of the clinical studies using a combination of a treatment with Bcl-2 antisense oligonucleotide followed by conventional therapies have been encouraging, particularly for chemotherapeutic agents, further investigations are warranted. However a persistent problem has been the limited ability to deliver sufficiently large doses of oligonucleotides to target cells in a manner that is medically tolerable, convenient and cost effective.

### SUMMARY OF THE INVENTION

In one aspect, the present invention provides a method of treating a patient with a proliferative disorder in need of treatment thereof, comprising administering to the patient, in each cycle of treatment, an effective amount of a pre-treatment; and, thereafter administering to the patient an effective amount of an oligomer. In another aspect of the invention the proliferative disorder is a neoplastic disease or an autoimmune disease. The neoplastic disease can be a cancer, a solid tumor or a hematologic malignancy. The pretreatment is administered prior to the administration of the oligomer and in preferred embodiments comprise one or more of radiotherapy, antineoplastic therapy including chemotherapy, acoustic therapy and thermal therapy. In one embodiment the oligomer or antisense oligonucleoside is selected from one or more of an antisense oligonucleotide, siRNA, microRNA, aptamer, morpholino, decoy molecule, and ribozyme. The oligomer can be directed to inhibiting the expression of a gene that is over-expressed in a cell such as a tumor cell, thereby causing or contributing to a proliferative disorder. Such genes include but are not limited to oncogenes, anti-apoptosis genes, transcription activators, protein kinases, genes that are involved in the signal transduction pathways, metabolic pathways, and genes that regulate cell growth and cell death.

In another aspect, the present invention provides a method of treating a cell to inhibit the expression of an oncogene, for example an anti-apoptotic gene. In one preferred embodiment the gene is the Bcl-2 gene, the c-Myb gene or the c-Myc gene. In yet another embodiment the antisense oligonucleotide is oblimersen. In yet another embodiment, the antisense oligonucleotide is G4460 or INX-3001, an antisense oligonucleotide that hybridizes to c-Myb mRNA (Genta, Incorporated, Berkeley Heights, N.J.).

In yet another aspect the present invention provides a method of treating a patient with a proliferative disorder in need of treatment thereof, comprising administering to the patient an effective amount of an oligomer wherein the oligomer is administered intermittently. In one preferred embodiment at least one day elapses between each dose of oligomer. In another embodiment at least two days, more preferably at least 3 days and even more preferably at least 5 days elapse between each dose of oligomer. In other embodiments the oligomer is administered about every week, about every two weeks or about every 3 or 4 weeks.

Another aspect of the present invention is a method for treating a human patient with cancer in need of treatment thereof comprising one or more treatment cycles in which the patient is first administered an effective amount of a pre-treatment and thereafter is administered an effective amount of an oligomer. In a preferred embodiment the pre-treatment is radiotherapy and the oligomer is oblimersen. In another embodiment the oligomer is administered no more frequently than about every other day, more preferably no more frequently than about every third day. In one aspect of the invention the oligomer is administered about every week, about every 2 weeks or about every 3 weeks. In another aspect of the invention the oligomer is administered at least one day, preferably at least 2 days, more preferably at least three days after the first pre-treatment dose of a cycle. In yet another aspect of the invention the neoplastic disease is lung cancer, breast cancer, prostate cancer, melanoma, chronic myeloid leukemia or lymphoma.

Another aspect of the present invention provides a method for treating a human patient with cancer in need of treatment thereof comprising one or more treatment cycles in which the patient is administered about 1 to 50 mg/kg, more preferably about 5 to 30 mg/kg, more preferably about 7 to 25 mg/kg and most preferably about 10 to about 30 mg/kg per dose of oligomer and the oligomer is administered no more frequently than once every other day, more preferably no more frequently than once every third day. In another embodiment the oligomer is oblimersen. In another embodiment the oligomer is administered in an amount that provides a mean peak plasma concentration of at least about 6 μg/mL, more preferably at least about 8 μg/mL, more preferably at least about 10 μg/mL. In yet another embodiment the mean peak plasma concentration is at least about 10 μg/mL, more preferably about at least 15 μg/mL and most preferably at least about 20 μg/mL. In another embodiment the oligomer is oblimersen and is administered in an amount that provides a mean peak plasma concentration of at least about 6 μg/mL, more preferably at least about 8 μg/mL, more preferably at least about 10 μg/mL. In yet another embodiment the mean peak plasma concentration is at least about 10 μg/mL, more preferably about at least 15 μg/mL and most preferably at least about 20 μg/mL.

Another aspect of the present invention is the use of an oligomer for the manufacture of a medicament for use in combination with a pre-treatment for the treatment of a proliferative disorder. In a preferred embodiment the proliferative disorder is a neoplastic disease. Another aspect of the present invention is the use of an oligomer for the manufacture of a medicament for use in combination with a pre-treatment for the treatment of a proliferative disorder, wherein said oligomer is administered intermittently. In a preferred embodiment the pre-treatment is selected from radiotherapy, chemotherapy, acoustic therapy, biologic therapy, thermal therapy or combinations thereof.

Another aspect of the present invention is the use of an oligomer for the manufacture of a medicament for the treatment of a proliferative disorder wherein the oligomer is administered intermittently. Yet another aspect of the present invention is the use of an anti-Bcl-2 antisense oligonucleotide for the manufacture of a medicament for use in combination with a pre-treatment which is radiotherapy for the treatment of a proliferative disorder, wherein said anti-Bcl-2 antisense oligonucleotide is administered intermittently. In a preferred embodiment the Bcl-2 oligonucleotide is oblimersen.

Another aspect of the present invention is the use of oblimersen for the manufacture of a medicament for use in combination with a pre-treatment which is radiotherapy for the treatment of a neoplastic disease, wherein the oblimersen is administered intermittently to achieve a mean peak plasma concentration of at least about 6 μg/mL. In a preferred embodiment the oblimersen is administered intermittently to achieve a mean peak plasma concentration of at least about 8 μg/mL, more preferably at least about 10 μg/mL, more preferably at least about 15 μg/mL. Another embodiment of the present invention is the use of oblimersen for the manufacture of a medicament for use in combination with a pre-treatment which is radiotherapy for the treatment of a neoplastic disease, wherein the oblimersen is administered intermittently to achieve a mean peak plasma concentration of at least about 20 μg/mL.

Another aspect of the present invention is the use of an anti-Bcl-2 antisense oligonucleotide which is oblimersen for the manufacture of a medicament wherein said anti-Bcl-2 antisense oligonucleotide is administered intermittently in an amount of from about 5 to about 50 mg/kg per dose. A preferred embodiment of the present invention is the use of an anti-Bcl-2 antisense oligonucleotide which is oblimersen for the manufacture of a medicament wherein said oblimersen is administered intermittently in an amount of from about 10 to about 50 mg/kg per dose. Another preferred embodiment of the present invention is the use of an anti-Bcl-2 antisense oligonucleotide which is oblimersen for the manufacture of a medicament wherein said oblimersen is administered intermittently in an amount of from about 15 to about 50 mg/kg per dose.

Another aspect of the invention is a method of treating a patient daily or every other day following pre-treatment via a subcutaneous or intravenous administration of oblimersen in an amount of less than 3 mg/kg, or less than 2 mg/kg, or less than 1 mg/kg when concomitant therapy is administered daily or up to 5 days a week.

### BRIEF DESCRIPTION OF FIGURES

FIG. 1A shows the dose and scheduling timeline for administration of fluorescent G3139 in mice bearing xenograft tumor.

FIG. 1B shows uptake of fluorescent-labeled G3139 oligonucleotides, FAM-G3139, a Bcl-2 antisense oligonucleotide in xenograft tumor tissue of mice treated IP with 5 mg FAM-G3139/kg (OBL5, low dose) daily for 7 days (left panel), and 15 mg FAM-G3139/kg (OBL15, high dose) intermittently at 2 day-intervals (day 1, 4 and 7). The tumor tissue is examined for uptake of the FAM-G3139 on day 8 (middle panel) and on day 12 (right panel).

FIG. 2A shows the dose and scheduling timeline for administration of 5 mg/kg fluorescent-labeled G3139 oligonucleotide, FAM-G3139 (OBL5) and X-rays in mice bearing xenograft tumor, where the X-rays (XRT) is administered on the first day of therapy, prior to administration of G3139 or on the last day of therapy, prior to the final administration of G3139.

FIG. 2B shows uptake of fluorescent G3139 oligonucleotide, FAM-G3139 in xenograft tumor tissue of mice treated intravenously with 5 mg FAM-G3139/kg daily for 7 days where X-ray is administered first (left panel) and (B) X-ray is administered last (right panel). The tumor tissue is examined for uptake on day 11.

FIG. 3(A) shows the dose and scheduling timeline for administration of 15 mg/kg fluorescent-labeled G3139 oligonucleotide, FAM-G3139 (OBL15) and X-rays in mice bearing xenograft tumor, where the X-rays (XRT) is administered first prior to administration of G3139 or last, after the final administration of G3139.

FIG. 3(B) shows uptake of fluorescent-labeled G3139 oligonucleotide, FAM-G3139 in xenograft tumor tissue of mice treated intravenously with 15 mg FAM-G3139/kg (OBL15) intermittently at 2-day intervals (day 1, 4 and 7) where X-ray is administered first (left panel) and (B) X-ray is administered last (right panel). The tumor tissue is examined for uptake on day 11.

FIG. 4 shows the long term persistence of fluorescent-labeled G3139 oligonucleotide, FAM-G3139 following uptake of the oligonucleotide in xenograft tumor tissue of mice having first X-ray pre-treatment on the right flank bearing tumor followed by a daily intravenous dose of 6 mg/kg FAM-G3139 for 5 days (top left panel); three doses of 10 mg/kg FAM-G3139 every other day (i.e. on day 1, 3, 5) (top middle panel); and a single dose of 30 mg/kg FAM-G3139 on the same day of X-ray pre-treatment (top right panel). Mice were sacrificed on day 8 for all groups and the tumor evaluated for uptake of FAM-G3139. FAM-G3139 uptake was compared to control tumor in the left flank of each of the mouse that are not irradiated and treated with FAM-G3139. [bottom panels].

FIG. 5 shows the quantitation of FAM-3139 in tumors of mice in FIG. 4.

FIG. 6 shows the experimental schedules and doses of G3139 administered to A549 NSCLC xenograft-bearing mice. The doses used were 2.5, 5, 7.5, 10, 20, 30, and 40 mg/kg administered either daily, on every other day, or at two- and three-day intervals.

FIG. 7 shows the tumor growth curves using the schedules and doses depicted in FIG. 8.

FIG. 8 shows the percent survival of A549 NSCLC xenograft bearing mice following administration of the various doses using the various doses depicted in FIG. 8.

### DETAILED DESCRIPTION OF THE INVENTION

The present invention provides methods and compositions for treating a human patient with a proliferative condition including auto-immune diseases and neoplastic diseases including cancer, hematologic malignancies and solid tumors by the administration of a oligomer administered intermittently with or without a pretreatment. It will be apparent to one of skill in the art that the terms cancer, tumor, neoplastic disease or neoplasm may be used interchangeably to refer to both solid tumors and to hematologic malignancies. Another aspect of the present invention is the treatment of a proliferative condition by the administration of one or more cycles of treatment wherein each cycle comprises a pre-treatment comprising one or more of radiotherapy, chemotherapy, acoustic therapy and biologic therapy that precedes the administration of an oligomer in that cycle. Such a pre-treatment includes a treatment that will provide improved uptake of the oligomer by cells exposed to the pre-treatment. A preferred embodiment is directed to the treatment of an oncogene-related disease such as cancer by means of one or more antisense oligomers, including but not limited to a Bcl-2 antisense oligonucleotide, administered in combination with a pre-treatment, such radiotherapy, thermotherapy, or acoustic therapy, whereby the pre-treatment is administered prior to the antisense oligomer and the antisense oligomer is administered in an intermittent dosing schedule that provides improved delivery to and treatment of diseased cells. The term oligomer, nucleotide oligomer or antisense oligomer and antisense oligonucleotide should be understood to include antisense oligonucleotides, siRNA, microRNA, aptamers, morpholinos, molecular decoys and derivatives thereof. As used herein, the term “derivative” refers to any pharmaceutically acceptable homologues, analogues, or fragments, naturally occurring or synthetic, corresponding to the pharmaceutical composition of the invention. Such a pre-treatment should be administered in an effective amount, namely sufficient to provide improved uptake of oligomer by cells exposed to the pretreatment, and preferably in a amount sufficient to have an effect on the disease without administration of an oligomer. An effective amount will be understood to include amounts of the pre-treatment routinely administered in the treatment of the proliferative condition. Similarly, an effective amount of oligomer is an amount sufficient to reduce the amount of the targeted molecules of interest.

Nucleotide Oligomers and Other Inhibitors of the Expression of a Gene of Interest

“Oligomers” is a general term as described above but also refers to short fragments of nucleic acids that hybridize to, and are thus complementary, to a portion of gene or its mRNA that is over-expressed in a diseased cell that is to be inhibited. Examples of diseased cells, include but are not limited to neoplastic or tumor cells and cells responsible for other proliferative diseases such as autoimmune disease. Genes that are over-expressed in tumor cells include but are not limited to oncogenes, anti-apoptosis genes, transcription activators, protein kinases, genes that are involved in the signal transduction pathways, and genes that regulate cell growth and cell death. Expression of oncogenes results, or is associated with, in increased frequency of malignancy. In addition, cells have developed ways of surviving natural cell death or injury caused by radiotherapy or other antineoplastic agents and treatments by the upregulation of certain genes such as anti-apoptosis genes, which can also result in the development of malignancy or the development of treatment resistant disease. The protein products of such genes inhibit apoptosis by binding to and inactivating the proteins that regulate cell death such as capases, p53, BAD, BAX and other cell death agonist. Thus the antisense oligomers contemplated in the present invention include nucleotide oligomers that hybridize to oncogenes, anti-apoptosis genes, and genes related to the family of protein kinases, transcription activators, and regulators of cell growth. In one embodiment of the invention, the antisense oligomers contemplated are directed to oncogenes, such as but are not limited to c-Myb, c-Myc, ErbB, Jun, Src, TGF-β, and MCC. In another embodiment, the antisense oligomer are antisense oligonucleotide of the anti-apoptosis gene families such as Bcl-XL, MDM-2, IAPs (Inhibitors of Apoptosis Proteins) such as cIAP1, cIAP2 and x-linked IAP (XIAP), Survivin, Bfl-1, LMP1 including homologues, analogues and derivatives thereof. In a further embodiment, the antisense oligomer are directed to genes involved in the signal transduction pathway, such as FADD, TRADD and TRAFF. In a preferred embodiment, the antisense oligomer is a Bcl-2 antisense oligomer.

Small interfering RNAs (siRNAs) are short double-stranded RNAs (dsRNAs) of about 21 nucleotides in length, having a 2 -3 nucleotide overhang at either end. Thus, each strand of the molecule has a phosphate group at its 5′ end and a hydroxyl group at its 3′ end. Like the antisense RNAs, siRNAs have been found to interfere with the expression of genes at the transcriptional level and are therefore useful for silencing specific genes of interest, particularly those that are cancer-related. Although siRNA are formed naturally in cells as a result of cleavage by an enzyme (the Dicer enzyme) that converts either long dsRNA or hairpin RNAs into siRNAs, synthetic siRNAs can be specifically designed to target any gene of interest. However, because of the overhangs at either ends, siRNA have very short half lives when artificially introduced into cells. As such, a further object of the present invention is to provide a method for an efficacious delivery and uptake of siRNA and hence enhanced treatment of a human patient with an anti-apoptotic-related disease using siRNA. In another embodiment, the invention provides a method for improved delivery and uptake of siRNA following pre-treatment, such as but not limited to radiotherapy at therapeutic or sub-therapeutic doses.

Micro-RNA or miRNA is a form of single-stranded RNA that is about 20-25 nucleotides in length and is thought to regulate expression of other genes at the level of translation. Unlike siRNAs, matured miRNAs are non-linear duplexes processed from a longer miRNA molecule that is transcribed from DNA. These RNA molecules are not translated into proteins but have shown to bind to mRNA of, for example, E2F-1 and inhibit the translation to the gene product such as E2F-1 protein, a protein that regulates cell proliferation. In one embodiment, the invention provides a method for improved delivery and uptake of miRNA, whereby the miRNA is substantially complementary to a portion of a pre-mRNA or mRNA that is related to proteins that regulate cell proliferation such as E2F-I. In another embodiment, the invention provides a method for improved delivery and uptake of miRNA following pre-treatment with cell injuring or inflammation causing agent, such as but not limited to radiotherapy at therapeutic or sub-therapeutic doses.

Aptamers are small molecules that can bind to other molecules. More specifically, aptamers can be classified as DNA or RNA aptamers. DNA or RNA aptamers are selective sequences that are capable of recognizing specific ligands by forming binding pockets. RNA or DNA aptamers can bind to nucleic acids, proteins, or small inorganic compounds. An example of a nucleic acid-based aptamer is the cyclic-AMP response element (CRE)-decoy aptamer (Genta Incorporated, Berkeley Heights, N.J.). The CRE-decoy aptamer binds to and blocks protein complexes that normally turn on genes which are regulated by CRE, thereby inhibiting tumor cell growth.

In one embodiment, the invention provides a method for improve delivery and uptake of a nucleic acid aptamer, wherein the aptamer is designed to specifically inhibit the gene at either the transcriptional level or translational level. In another embodiment, the invention provides a method for improved delivery and uptake of a nucleic acid aptamer, wherein the aptamer specifically binds a protein that regulates cell proliferation or apoptosis, and is administered at intermittent intervals following administration of a pre-treatment including but not limited to radiotherapy.

### Pre-Treatment

One aspect to the present invention relates to one or more treatment cycles that comprise a pre-treatment before administration of an oligomer-based treatment. The pre-treatment includes one or more of a number of more conventional therapies as detailed below:

Radiotherapy: Radiotherapy is one of the primary therapies for treating cancers, particularly in the treatment of cancers, such as breast cancer, lung cancer and prostate cancers. (For a review on radiotherapy, see, De Vita, Jr, et al., Cancer: Principles & Practice of Oncology, J.B. Lippincott Company (Publisher), 3^{rd} Edition; in Chapter 15—Principle of Radiation therapy). For radiation alone or in combination with conventional chemotherapy to be an effective therapy, the patient is often treated with large doses of radiation. Although cure rates through radiation therapy have increased as a result of sophisticated planning and advanced delivery methods for high doses of radiation, treatment failures still occur. Commonly radiation therapy is used in combination with other anti-neoplastic agents, such as chemotherapeutic drugs, antibodies, or hormones. In certain types of cancer, resistance to radiation and anti-neoplastic agents occurs due to over-expression of oncogenes that result in the inhibition of the apoptotic pathways that normally would be activated after injury to cells caused by radiotherapy or other antineoplastic agents. For example, over-expression of Bcl-2, an inhibitor of apoptosis is known to play a role in many types of cancer, including melanoma, chronic myeloid leukemia and others. As such, inhibiting Bcl-2 function may enhance sensitivity to radiation in tumor cells.

Previously, all treatment regimens utilizing an antisense oligomer in combination with other antineoplastic treatments have required the initial administration of the antisense oligomer, believed to be necessary to inhibit the expression of the anti-apoptotic gene, before the administration of the antineoplastic treatment. Clinical studies using antisense oligonucleotide therapy, such as Bcl-2 antisense oligonucleotides, have been conducted whereby the oligonucleotides are administered daily, typically via continuous IV infusion for 10 days or by continuous subcutaneous infusion for 14 days prior to treatment with conventional therapies. (See, G. Marcucci, et al. (2003) Blood 101:425-432; and J. S. Waters, et al. (2000) J. Clin. Oncol. 18:1812-1823). The rationale for such a scheduling strategy has been the belief that it is necessary to ensure that local levels of the oligonucleotide in the cell are sufficiently high prior to administering more conventional therapy in order to readily inhibit expression of the Bcl-2 gene and thereby prevent the cell from surviving the injury caused by treatment with, for example, radiation or an antineoplastic agent. The results of these studies have been encouraging but not optimal.

Synthesis of clinical grade oligonucleotides under GMP requirements is often costly and as such, administration of large quantities of oligonucleotides, particularly on the currently used daily/continuous schedule can be economically prohibitive for many patients and/or health insurances. In one embodiment the present invention provides a more cost effective method of treatment.

Access to the cytoplasm is a key issue because the oligonucleotide must be able to redistribute into the cell where it is able to hybridize with the mRNA to inhibit expression of gene product with anti-apoptosis function. Although a natural uptake mechanism is not presently known, it has been thought that oligonucleotide may gain access to the cytoplasm either by passive diffusion or endosomal leakage. However, evidence for uptake by passive diffusion, particularly with charged oligonucleotide is weak and healthy cells are not normally leaky. Furthermore, pharmacokinetic studies in Phase I clinical trials have demonstrated that the mean plasma half-life for elimination of the oligonucleotide are about 30 minutes to about 8 hours depending on the dose and mode of administration. Until now the ability to deliver oligomer to tumor cells has been considered to be a function of the duration that the tumor cells are exposed to circulating oligomers, with treatment strategies being directed to providing as long an exposure as possible, hence the continuous and daily administration schedules. It remains a critical issue in the field to optimize the uptake of oligonucleotides in tumor cells in order to provide maximal therapeutic value that is not only effective but also economical.

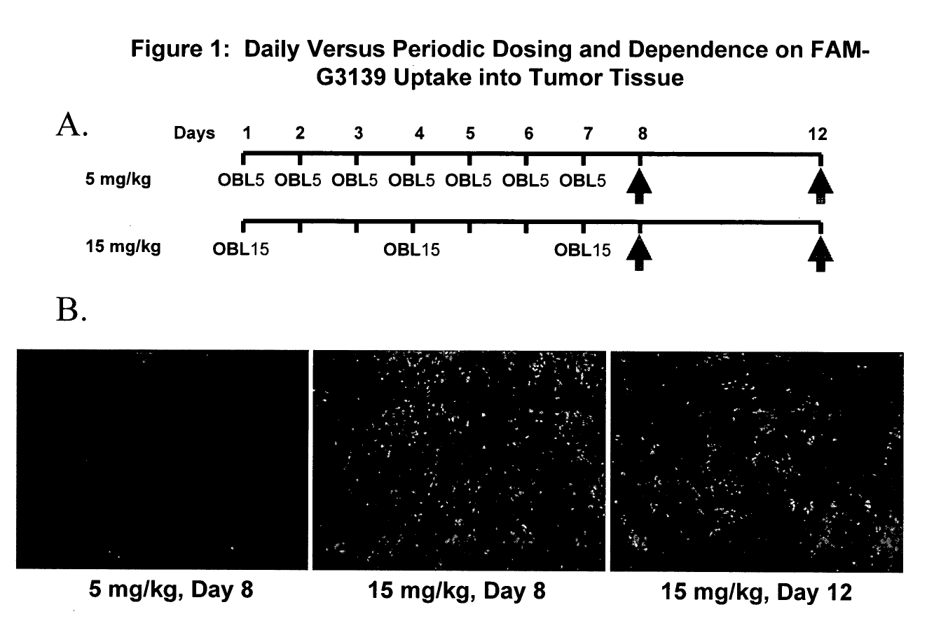

The present invention is based in part on the surprising discovery that high doses of an anti-apoptotic oligonucleotide against Bcl-2 administered at intermittent intervals provide higher uptake of the oligonucleotide by tumor cells in mouse xenograft than when such animals are treated in the conventional manner with daily administration of oligonucleotide. Intermittent dosing of xenograft tumor-bearing mice with fluorescent-labeled Bcl-2 antisense oligonucleotide at a dose of 15 mg /kg on days 1, 4, and 7 (3-day interval intermittent dosing schedule) showed significantly improved uptake and retention of labeled oligonucleotide even five days post-treatment compared to daily dosing. (See FIG. 1B). This increased uptake unexpectedly has been shown to be further enhanced by administering a pre-treatment, such as radiotherapy to the tumor mass prior to administration of the Bcl-2 antisense oligonucleotide. Best results were shown when the animal first received a pre-treatment and thereafter the oligomer was administered intermittently rather than on the conventional continuous/daily schedule.

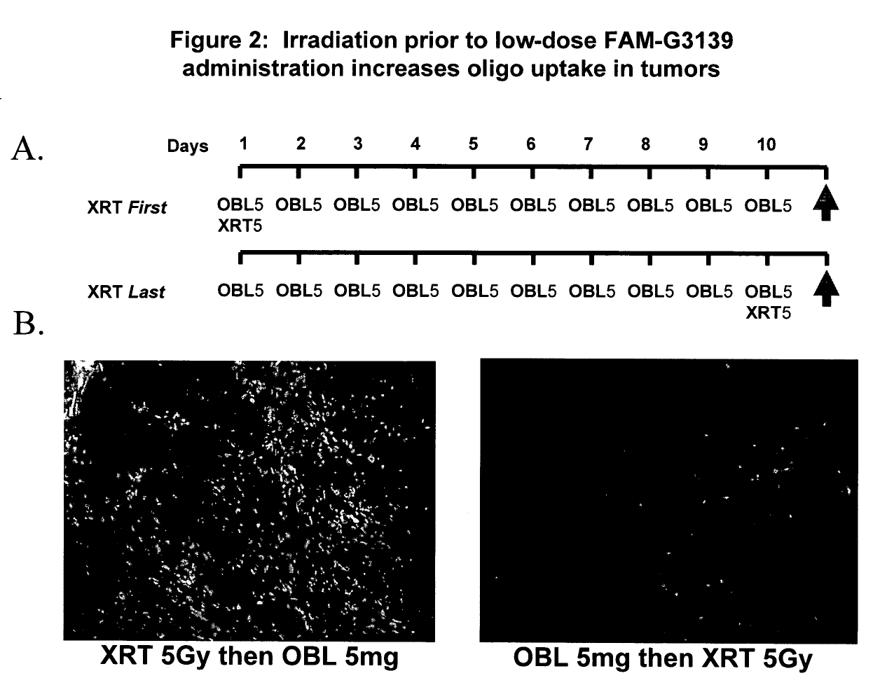

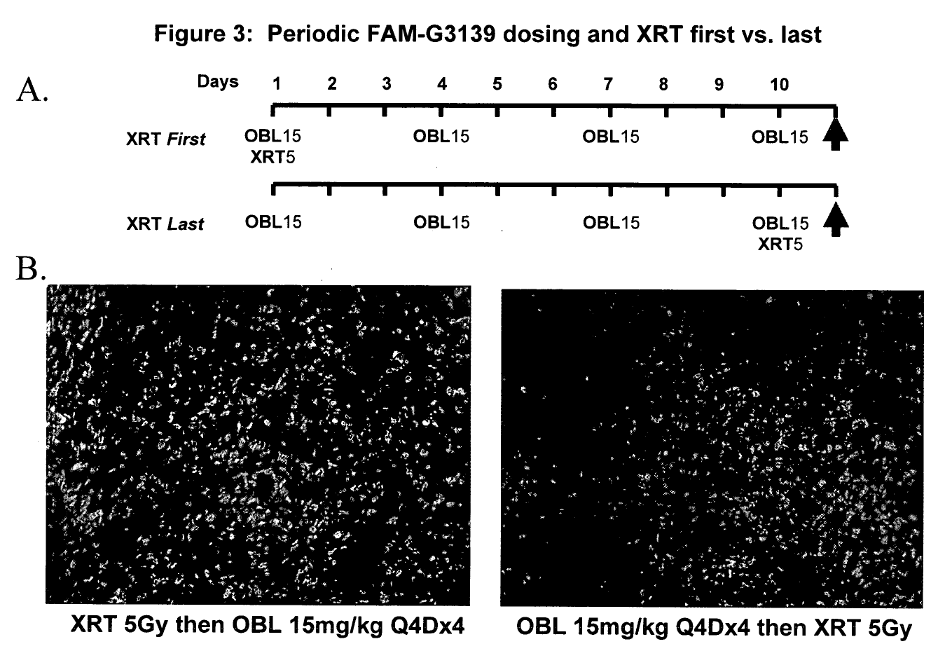

It surprisingly was found that when, contrary to current practice, radiotherapy (5 Gy of X-Ray) was administered on day 1 preceding a ten-day Bcl-2 antisense treatment regimen of a daily dose of 5 mg/kg of oligonucleotide, uptake of fluorescent-labeled Bcl-2 antisense oligonucleotide was greatly enhanced compared to when the same dose of radiotherapy was administered on day 10, after a 10 day treatment of a daily dose of 5 mg/kg of oligonucleotide (See, FIG. 2B, left panel) or when no radiotherapy was administered (See, FIG. 2B, right panel). Moreover, pre-treatment with radiation followed by intermittent dosing of the same total dose of Bcl-2 antisense oligonucleotide on days 1, 4, 7 and 10 (3-day interval intermittent dosing schedule for a total of 4 doses of 15 mg/kg each) also resulted in significantly improved uptake. (See, FIG. 3B). With the oligomer intermittent treatment regimen, although uptake was also increased compared to daily dosing in tumors irradiated after the last Bcl-2 antisense oligonucleotide treatment on day 10, the greatest uptake was observed when the tumor was pre-treated with radiation on day 1, prior to administration of the oligomer.

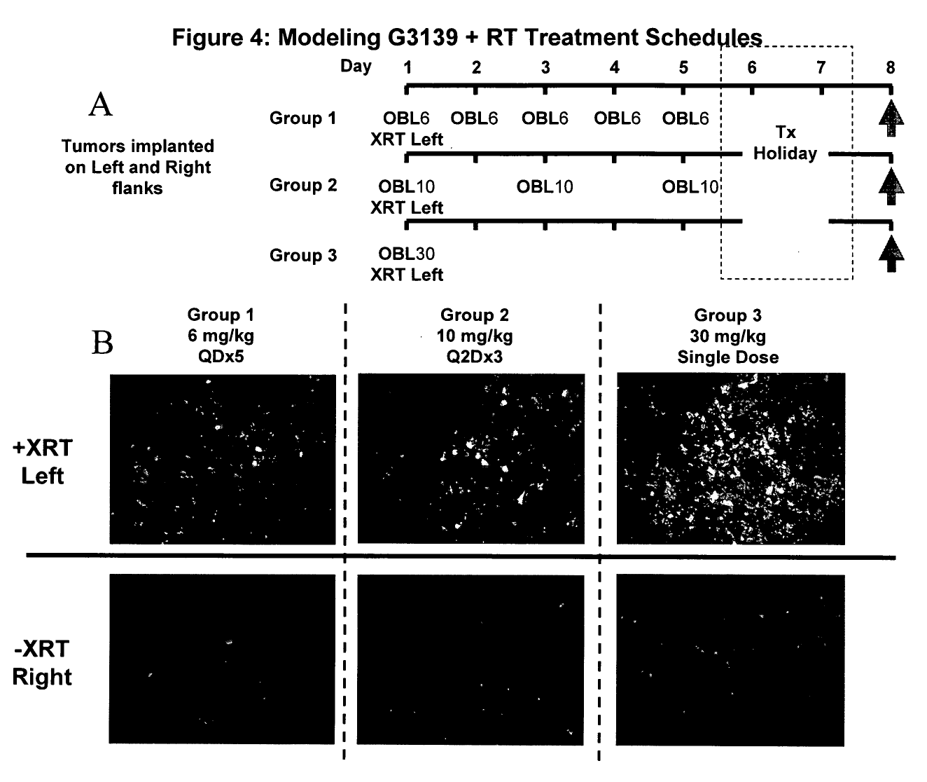

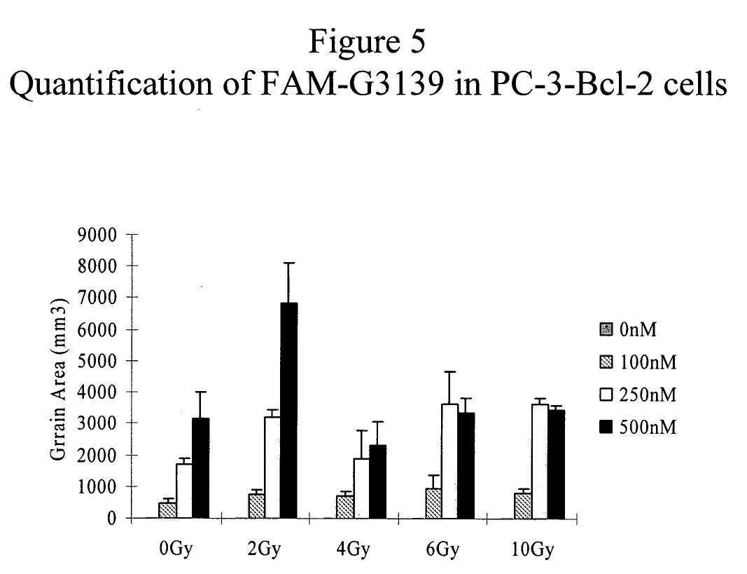

In addition to the surprising observation that pre-treatment with radiotherapy provided enhanced uptake to the oligomer, as did intermittent treatment with oligomer, with the highest uptake being seen with pre-treatment with radiation followed by intermittent treatment with the oligomer, it unexpectedly was found that pre-treatment with radiation before the initiation of treatment with the oligomer also provided improved/prolonged retention of the oligomer within the cells pretreated with radiation. Substantial amounts of oligomer were detectable within the cells for at least five to seven days post treatment. Moreover, those animals receiving a single large dose of oligomer after radiation therapy had greater retention then those who received intermittent dosing, which in turn had greater retention than those animals treated with daily doses of oligomer. In all cases, the retention was substantially greater in the animals that had received the pre-treatment. Specifically, the oligonucleotide persisted for at least up to five to seven days after administration of radiotherapy on day 1 and administration of a single high dose of 30 mg/kg oligonucleotide on the same day as, but after, the radiation compared to mice that have been pre-treated with radiotherapy followed by administration of 10 mg/kg of oligonucleotide every other day (days 1, 3, and 5) or daily administration of 6 mg/kg for 5 days. (See FIG. 4 and FIG. 5).

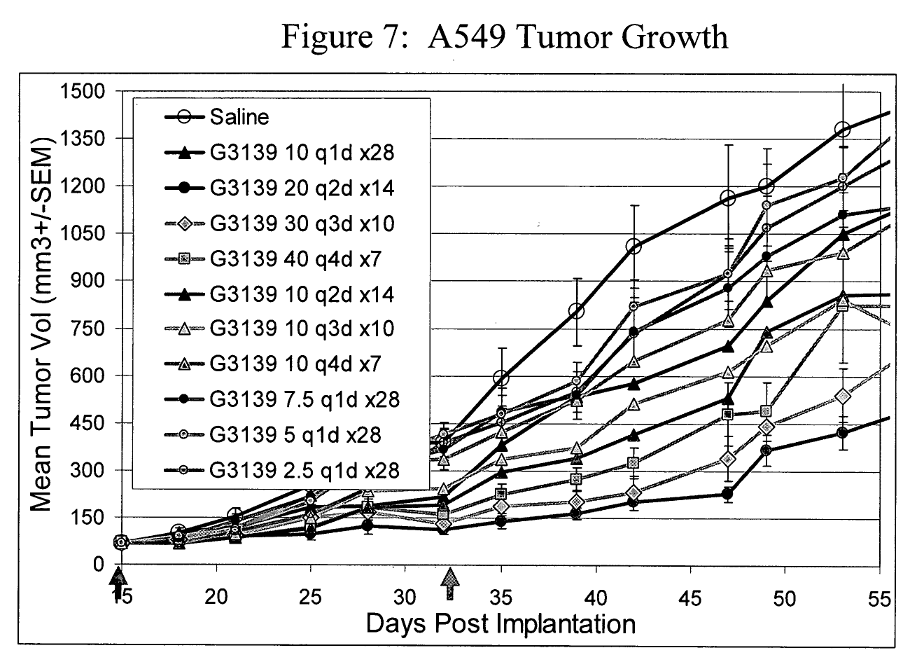

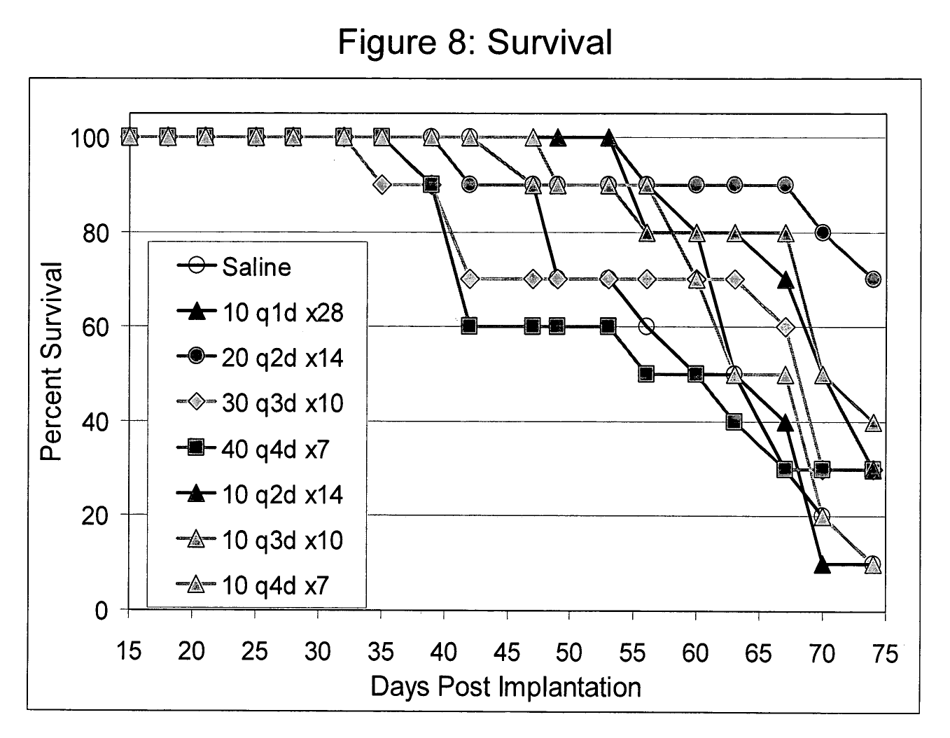

The results from these studies demonstrate that using a pre-treatment such as radiotherapy not only provides improved uptake and retention of oligonucleotides, but also provides a method of treatment that provides improved benefit optionally using smaller quantities of oligonucleotide administered in a more convenient manner. By providing improved uptake and prolonged retention of the antisense oligonucleotides, this method of treatment provides an improved means of inhibiting the expression of anti-apoptotic genes and thereby improved treatment of such a condition. (See, FIG. 7 and FIG. 8) These studies provide a method for an improved clinical application of antisense nucleotide therapy in cancer therapy. Furthermore, less frequent dosing with or without lower amounts of oligonucleotides provides less frequent visits to the treatment centers, saving both time and expenses.

Based on the surprising results discussed above, the present invention therefore contemplates a treatment regimen comprising one or more cycles of therapy comprising administering one or more pre-treatments each cycle prior to administration, preferably at intermittent intervals, of high or low doses of oligomer, such as but not limited to Bcl-2 antisense oligonucleotide. Such a treatment regimen would be administered in cycles, optionally until the desired result is attained, a predefined number of cycles has been administered or until the treating physician determines that a change in treatment is appropriate. The present invention also contemplates a method for improving/enhancing delivery of a composition that inhibits the expression of an anti-apoptotic gene by administering one or more pre-treatments prior to intermittent treatment with high doses of the composition. The pre-treatment relates to a treatment that is capable of damaging the cell membrane and/or causing an inflammatory response. In one embodiment of the present invention, selection of a pre-treatment is based on the ability to control the extent and severity of damage to the cell and/or the inflammatory response. The pre-treatment can be a physical, a biologic or a pharmacological treatment. An example of a physical pre-treatment is electromagnetic radiation, such as but not limited to X-rays, gamma rays, beta particles, ultraviolet rays, infra-red rays, radiowaves, and microwaves. Another embodiment of radiotherapy is a proton beam or an electron pulse.

X-ray or gamma ray are two of the most common radiotherapies used for cancer treatment and remains a primary curative modality for malignant tumors. However, these agents have not been considered by clinicians as a method for enhancing uptake of oligomers. One of the advantages of using X-ray or gamma ray is the ability of radiation to penetrate deep into the body cavity and to direct it to a specific site without being physically invasive or systemic, although the present invention also contemplates the use of such treatment modalities as total body irradiation (“TBI”), skin irradiation for conditions such as cutaneous lymphomas and brachytherapy in appropriate circumstances that would be readily apparent to one of ordinary skill in the art.

In one embodiment of the invention, the electromagnetic radiation is a beta particle. In another embodiment of the invention, the electromagnetic radiation is a radiowave or a microwave. In a preferred embodiment of the invention, the electromagnetic radiation is a gamma ray and in a more preferred embodiment, the electromagnetic radiation is an X-ray.

The electromagnetic radiation can be administered to a patient, conventionally, stereotactically or as whole body radiation. Stereotactic radiation is a precise method of delivering radiation to a tumor with sparing of the surrounding normal tissue. The radiation can be administered as a single dose, in conventional fractions or as hyperfractionated radiation.

In one embodiment of the invention, radiotherapy is given to the patient, stereotactically or conventionally, prior to treatment with an oligomer. In another embodiment of the invention, radiotherapy is given stereotactically followed by intermittent administration of the oligomer from about 1 hour to about 24 hours or longer after pre-treatment. Such pre-treatment can be given over several days or even weeks during which time oligomer is administered, provided that the pretreatment is initiated prior to the oligomer treatment.

In a more preferred embodiment of the invention, radiotherapy is given, stereotactically or conventionally, followed by intermittent administration of the oligomer from about 1 to about 10 days post pre-treatment. For example, in one embodiment of the invention, the oligomer is administered no more frequently than every other day. In a further embodiment, the oligomer is administered every two, three, or four days. In a preferred embodiment, the oligomer is administered every five days. In a more preferred embodiment of the invention, the oligomer is administered weekly, biweekly, monthly, or bimonthly. In a further preferred embodiment, the oligomer is administered once after the pre-treatment on the same day as the pre-treatment or a period of about 1 to about 7 days post pre-treatment.

In another embodiment of the invention, radiotherapy is given to the patient in a stereotactic manner prior to treatment with an oligomer. In another embodiment of the invention, radiotherapy is given in a stereotactic manner followed by intermittent administration of the oligomer or inhibitor at about 3 hours to about 24 hours after pre-treatment. In a more preferred embodiment of the invention, the radiotherapy is given in a fractionated manner followed by intermittent administration of the oligomer at about 2 to about 10 days post pre-treatment.

In a further preferred embodiment, the radiotherapy is given in a stereotactic manner followed by immediate treatment with an oligomer followed by intermittent dosing at about 2 to about 10 days intervals. For example, in one embodiment of the invention, the oligomer is administered no more frequently than every other day. In a further embodiment, the oligomer is administered every two, three, or four days. In a preferred embodiment, the oligomer is administered every five or six days. In a more preferred embodiment of the invention, the oligomer is administered weekly, biweekly, monthly or bimonthly.

The amount of radiation administered as pre-treatment will be readily apparent to one of ordinary skill in the art. Dosing is dependent on the volume, type and shape of the tumor. In addition, the proximity of normal structures or tissues must also be considered. In one embodiment, the amount of radiotherapy to be administered as a pre-treatment is an amount that is not conventionally considered to be a therapeutic dose. The amount of such a dose also will be readily apparent to one of skill in the art and optionally may be used in an area that previously has been treated, or unable to tolerate the administration of greater conventionally therapeutic amounts of radiation. In another embodiment the pre-treatment is administered in a dose and manner that is considered to be a therapeutic amount if used alone or in combination with chemotherapy or other commonly used antineoplastic agents.

In a further embodiment, the present invention contemplates a method for reducing the amount of oligomer administered in a cycle of therapy by first administering the pre-treatment followed by administering one or more doses of oligomer whereby the oligomer is administered from about once every other day to about once every 7 days.

Acoustic therapy: The present invention also contemplates the utilization of low- or high-intensity focused ultrasound as a non-invasive pre-treatment for the patient with cancer, prior to administration of the oligomer. Like radiotherapy, acoustic therapy provides a non-invasive method for penetrating deep into the body cavity to the specific site, minimizing damage to normal tissue.

In one embodiment of the invention, pre-treatment with low- or high-intensity ultrasound is administered prior to treatment with oligomer. In another embodiment of the invention, low- or high-intensity ultrasound is given followed by intermittent administration of the oligomer at about 1 hour to about 24 hours after the pre-treatment. In a more preferred embodiment of the invention, a human patient with cancer is provided with a dose of low- or high-intensity ultrasound followed by intermittent administration of oligomer from about 2 to about 10 days post pre-treatment. For example, in one embodiment of the invention, the oligomer is administered no more frequently than every other day. In a further embodiment, the oligomer is administered about every two, three, or four days. In a preferred embodiment, the oligomer is administered every five days. In a more preferred embodiment of the invention, the oligomer is administered about weekly, biweekly, monthly, or bimonthly.

The dose of ultrasound administered as a pre-treatment is readily apparent to those skilled in the art. The intensity of ultrasound used is dependent on the volume, type, and shape of the tumor. In addition, the proximity of normal structures or tissues in relation to the location of the tumor, i.e. whether the tumor is located in the deep recess of the body cavity or the surface of the skin, must also be taken into account. In one embodiment of the present invention energies of about 0.1 to about 3 W/cm² are used. In another embodiment of the invention, the intensity energy is about 3 to about 10 W/cm². In a further embodiment of the invention, the intensity energy is about 100 to about 1000 W/cm²

Thermotherapy: One form of thermotherapy is hyperthermia therapy, which is the application of high temperatures of up to about 45° C. to the body tissue, usually with minimal injury to the normal tissue.

It is envisioned that one embodiment would encompass applying local hyperthermia to a small area within the bulk of a tumor using various techniques to heat the tumor. Techniques include but are not limited, to microwave, radiofrequency, and ultrasound. Local hyperthermia may also include external approaches for tumors in or just below the skin, intra-luminal, or endocavitary methods for tumors within or near body cavities, interstitial methods for tumors localized deep within the body and means that provide isolated perfusion such as isolated limb perfusion means.

In yet another embodiment, regional hyperthermia may be used to heat large areas of the tumor tissue. In a further embodiment of the invention, whole body hyperthermia may be used to treat metastatic cancer that has spread throughout the body. The body temperature in the instant embodiment may be raised from about 41° C. to about 42° C.

In yet another embodiment, pre-treatment includes alternating applications of hyperthermia therapy locally, regionally or whole body.

In a preferred embodiment of the invention, the pre-treatment using hyperthermia is administered immediately prior to the administration oligomer with or without subsequent administration of the oligomer at intermittent intervals of about 2 to about 10 days. For example, in one embodiment of the invention, the oligomer is administered no more frequently than about every other day. In a further embodiment, the oligomer is administered about every two, three, or four days. In a preferred embodiment, the oligomer is administered every five days. In a more preferred embodiment of the invention, the oligomer is administered weekly, biweekly, monthly, or bimonthly.

In a more preferred embodiment of the invention, hyperthermia pre-treatment is applied from about 1 hour to 24 hours prior to the administration of oligomer, followed by subsequent administrations of the oligomer at intermittent intervals of about 2 to about 10 days. In one embodiment of the invention, the oligomer is administered no more frequently than every other day. In a further embodiment, the oligomer is administered every two, three, or four days. In a preferred embodiment, the oligomer is administered every five days. In a more preferred embodiment of the invention, the oligomer is administered weekly, biweekly, monthly, or bimonthly.

Pharmacologic therapy: Furthermore, the present invention also contemplates the use of pharmacological therapies as pre-treatment for a human patient with a proliferative disorder, including but not limited to cancer. These pharmacological agents are applied in an effective amount sufficient to pre-treat tumor cells prior to administration of oligomer. The effective amount will be readily determined by one of ordinary skill and includes but is not limited to conventionally therapeutic doses of the pharmacological agent or sub-therapeutic amounts. Examples of pharmacological agents include but are not limited to chemoagents belonging to the class of anti-neoplastic agents, antibiotics, lipases, detergents, small molecules, agonists or antagonist of kinases, or its derivatives and analogues thereof. Classes of anti-neoplastic agents include but are not limited to alkylating agents, topoisomerase inhibitors, plant alkanoids and terpenoids, nucleotides/nucleosides analogues, and anti-metabolites. Some examples of anti-neoplastic agents contemplated include but are not limited to bortezomib, gemcitabine, imatinib, fludarabine, oxaliplatin, docetaxel, palcitaxel, thalidomide, 5-FU, doxorubicin, arabinoside-C, carboplatin, daunomycin, dexamethasone and etoposide.

In one embodiment of the present invention, a human patient with cancer is treated with one or more cycles of therapy comprising a pre-treatment prior to the administration of oligomer with or without subsequent administration of the oligomer at intermittent intervals of about 2 to about 10 days. For example, in one embodiment of the invention, the oligomer is administered no more frequently than every other day. In a further embodiment, the oligomer is administered every two, three, or four days. In a preferred embodiment, the oligomer is administered about every five days to about every 10 days. In a more preferred embodiment of the invention, the oligomer of the expression of the gene of interest is administered about weekly, biweekly, monthly, or bimonthly.

In another preferred embodiment of the invention, the pharmacological pre-treatment is administered about 1 to about 24 hours prior to the administration of oligomer followed by subsequent administrations of oligomer at intermittent intervals of about 2 to about 10 days. In one embodiment of the invention, the oligomer is administered no more frequently than every other day. In a further embodiment, the oligomer is administered every two, three, or four days. In a preferred embodiment, the oligomer is administered every five days. In a more preferred embodiment of the invention, the oligomer is administered about weekly, biweekly, monthly, or bimonthly.

Biological therapies: The present invention further provides for a pharmaceutical composition comprising one or more biological therapy as a pre-treatment. Examples of biologic pre-treatment include but not limited to antibodies, proteins such as perforin, proteases, hormones, cytokines, growth factors and prostaglandins.

In one embodiment of the invention, a human patient with cancer receives a pre-treatment with one or more biologic composition. The biologic pre-treatment composition may be at sub-therapeutic doses and may be applied immediately prior to the administration of inhibitors of a pre-treatment with or without subsequent administration of the oligomer at intermittent intervals of about 2 to about 10 days.

In another preferred embodiment of the invention, the biologic pre-treatment composition may be applied about 1 to 24 hours prior to the administration of an oligomer followed by subsequent administrations at intermittent intervals of about 2 to about 10 days. For example, in one embodiment of the invention, the oligomer is administered no more frequently than every other day. In a further embodiment, the oligomer is administered every two, three, or four days. In a preferred embodiment, the oligomer is administered about every five days to about every 10 days. In a more preferred embodiment of the invention, the oligomer is administered about weekly, biweekly, monthly, or bimonthly.

Inhibitors of the Expression of Anti-Apoptotic Genes

The invention contemplates use of one or more inhibitors of anti-apoptotic genes, including but not limited to Bcl-2 antisense oligonucleotide such as oblimersen (Genasense®; G3139), or its derivatives, analogues, fragments, hybrids, mimetics, and oncogenes thereof. As used herein, the term “derivative” refers to any pharmaceutically acceptable homolog, analogue, or fragment, naturally occurring or synthetic, corresponding to the pharmaceutical composition of the invention. Antisense oligonucleotides suitable for use in the invention include nucleotide oligomers which range in size from 5 to 10, 10 to 20, 20 to 50, 50 to 75, or 75 to 100 bases in length; preferably 10 to 40 bases in length; more preferably 15 to 25 bases in length; most preferably 18 bases in length. The target sequences to which the antisense nucleotide binds may be RNA or DNA that expressed protein at elevated levels in a diseased or cancer cell. The target sequences may be single-stranded or double-stranded. Target molecules include, but are not limited to pre-mRNA, MRNA, DNA, and proteins. In a one embodiment, the target molecule is mRNA. In a preferred embodiment, the target molecule is Bcl-2 pre-mRNA or Bcl-2 mRNA. In a specific embodiment, the antisense oligonucleotides hybridize to a portion anywhere along the Bcl-2 pre-mRNA or MRNA. The antisense oligonucleotides are preferably selected from those oligonucleotides which hybridize to the translation initiation site, donor splicing site, acceptor splicing site, sites for transportation, or sites for degradation of the Bcl-2 pre-mRNA or MRNA.

Several Bcl-2 antisense oligonucleotides have been assessed previously with variable results (See, e.g., SEQ. ID. NOS.:1-17 in U.S. Pat. No. 5,831,066). Examples of Bcl-2 antisense oligonucleotides that may be used in accordance with the present invention are described in detail in U.S. patent application Ser. No. 08/217,082, now U.S. Pat. No. 5,734,033; U.S. patent application Ser. No. 08/465,485, now U.S. Pat. No. 5,831,066; and U.S. patent application Ser. No.09/080,285, now U.S. Pat. No.6,040,181, each of which is incorporated herein by reference in its entirety.

In one embodiment, the Bcl-2 antisense oligonucleotide is substantially complementary to a portion of a Bcl-2 pre-mRNA or mRNA, or to a portion of a pre-mRNA or mRNA that is related to Bcl-2. In a preferred embodiment, the Bcl-2 antisense oligonucleotide hybridizes to a portion of the translation-initiation site of the pre-mRNA coding strand. In a more preferred embodiment, the Bcl-2 antisense oligonucleotide hybridizes to a portion of the pre-mRNA coding strand that comprises the translation-initiation site of the human Bcl-2 gene. More preferably, the Bcl-2 antisense oligonucleotide comprises a TAC sequence which is complementary to the AUG initiation sequence of the Bcl-2 pre-mRNA or RNA.

In another embodiment, the Bcl-2 antisense oligonucleotide hybridizes to a portion of the splice donor site of the pre-mRNA coding strand for the human Bcl-2 gene. Preferably, this nucleotide comprises a CA sequence, which is complementary to the GT splice donor sequence of the Bcl-2 gene, and preferably further comprises flanking portions of 5 to 50 bases, more preferably from about 10 to 20 bases, which hybridizes to portions of the Bcl-2 gene coding strand flanking said splice donor site.

In yet another embodiment, the Bcl-2 antisense oligonucleotide hybridizes to a portion of the splice acceptor site of the pre-mRNA coding strand for the human Bcl-2 gene. Preferably, this nucleotide comprises a TC sequence, which is complementary to the AG splice acceptor sequence of the Bcl-2 gene, and preferably further comprises flanking portions of 5 to 50 bases, more preferably from about 10 to 20 bases, which hybridizes to portions of the Bcl-2 gene coding strand flanking said splice acceptor site. In another embodiment, the Bcl-2 antisense oligonucleotide hybridizes to portions of the pre-mRNA or mRNA involved in splicing, transport or degradation.

One of average skill in the art can recognize that antisense oligomers suitable for use in the present invention may also be substantially complementary to other sites along the Bcl-2 pre-mRNA or mRNA, and can form hybrids. The skilled artisan will also appreciate that antisense oligomers, that hybridize to a portion of the Bcl-2 pre-mRNA or mRNA whose sequence does not commonly occur in transcripts from unrelated genes are preferable so as to maintain treatment specificity.

The design of the sequence of a Bcl-2 antisense oligonucleotide can also be determined by empirical testing and assessment of clinical effectiveness, regardless of its degree of sequence homology to, or hybridization with, the Bcl-2 gene, Bcl-2 pre-mRNA, Bcl-2 mRNA, or Bcl-2 related nucleotide sequences. One of ordinary skill in the art will appreciate that Bcl-2 antisense oligonucleotides having, for example, less sequence homology, greater or fewer modified nucleotides, or longer or shorter lengths, compared to those of the preferred embodiments, but which nevertheless demonstrate responses in clinical treatments, are also within the scope of the invention.

The antisense oligonucleotides may be RNA or DNA, or derivatives thereof. The particular form of antisense oligonucleotide may affect the pharmacokinetic parameters of the oligomer such as bioavailability, metabolism, half-life, etc. As such, the invention contemplates antisense oligonucleotide derivatives having properties that improve cellular uptake, enhance nuclease resistance, improve binding to the target sequence, or increase cleavage or degradation of the target sequence. The antisense oligonucleotides may contain bases comprising, for example, phosphorothioates or methylphosphonates. The antisense oligonucleotides, instead, can be mixed oligomers containing combinations of phosphodiesters, phosphorothioate, and/or methylphosphonate nucleotides, among others. Such oligomers may possess modifications which comprise, but are not limited to, 2-O′-alkyl or 2-O′-halo sugar modifications, backbone modifications (e.g., methylphosphonate, phosphorodithioate, phosphorodithioate, formacetal, 3′-thioformacetal, sulfone, sulfamate, nitroxide backbone, morpholino derivatives and peptide nucleic acid (PNA) derivatives), or derivatives wherein the base moieties have been modified (Eghoim, et al., 1992, Peptide Nucleic Acids (PNA)-Oligonucleotide Analogues With An Achiral Peptide Backbone, Arghya and Norden 2000, “Peptide Nucleic acid (PNA): its medical and biotechnical applications and promise for the future”, FASEB 14:1041-1060). In another embodiment, antisense oligonucleotides comprise conjugates of the oligonucleotides and derivatives thereof (Goodchild, 1990, “Conjugates of oligonucleotides and modified oligonucleotides: a review of their synthesis and properties”, Bioconjug. Chem. 1(3):165-87).

For in vivo therapeutic use, a phosphorothioate derivative of the Bcl-2 antisense oligonucleotide is preferable, at least partly because of greater resistance to degradation. In one embodiment, the Bcl-2 antisense oligonucleotide is a hybrid oligomer containing phosphorothioate bases. In another embodiment, the Bcl-2 antisense oligonucleotide contains at least one phosphorothioate linkage. In another embodiment, the Bcl-2 antisense oligonucleotide contains at least three phosphorothioate linkages. In yet another embodiment, the Bcl-2 antisense oligonucleotide contains at least three consecutive phosphorothioate linkages. In yet another embodiment, the Bcl-2 antisense oligonucleotide is comprised entirely of phosphorothioate linkages. Methods for preparing oligonucleotide derivatives are known in the art. See, e.g., Stein et al., 1988, Nucl, Acids Res., 16:3209-21 (phosphorothioate); Blake et al., 1985, Biochemistry 24:6132-38 (methylphosphonate); Morvan et al., 1986, Nucl. Acids Res. 14:5019-32 (alphadeoxynucleotides); Monia et al., 1993, “Evaluation of 2′-modified oligonucleotides containing 2′ deoxy gaps as antisense inhibitors of gene expression”, J. Biol. Chem. 268:14514-22 (2′-0-methyl-ribonucleosides); Asseline et al., 1984, Proc. Natl. Acad. Sci. USA 81:3297-3301 (acridine); Knorre et al., 1985, Biochemie 67:783-9; Vlassov et al., 1986, Nucl. Acids Res. 14:4065-76 (N-2-chlorocethylamine and phenazine); Webb et al., 1986, Nucl. Acids Res. 14:7661-74 (5-methyl-N⁴-N⁴-ethanocytosine); Boutorin et al., 1984, FEBS Letters 172:43-6 (Fe-ethylenediamine tetraacetic acid (EDTA) and analogues); Chi-Hong et al., 1986, Proc. Natl. Acad. Sci. USA 83:7147-51(5-glycylamido-1,10-o-phenanthroline); and Chu et al., 1985, Proc. Natl. Acad. Sci. USA 82:963-7 (diethylenetriaamine-pentaacetic acid (DTPA) derivatives).

The effective dose of Bcl-2 antisense oligonucleotide to be administered per dose ranges from about 0.1 to about 50 mg/kg/per dose, preferably from about 1 to about 50 mg/kg/per dose, more preferably from about 5 to about 30 mg/kg/per dose and most preferably from about 10 to about 30 mg/kg/per dose. The dose of Bcl-2 antisense oligonucleotide to be administered can be dependent on the mode of administration. For example, intravenous administration of a Bcl-2 antisense oligonucleotide would likely result in a significantly higher full body dose than a full body dose resulting from a local implant containing a pharmaceutical composition comprising Bcl-2 antisense oligonucleotide. In one embodiment, a Bcl-2 antisense oligonucleotide is administered subcutaneously at a dose of about 1 to about 50 mg/kg/per dose; more preferably at a dose of about 4 to about 30 mg/kg/per dose, most preferably at a dose of about 5 to about 15 mg/kg/per dose. In another embodiment, a Bcl-2 antisense oligonucleotide is administered intravenously at a dose of about 1 to about 50 mg/kg/per dose; more preferably at a dose of about 4 to about 30 mg/kg/per dose; most preferably at a dose of about 5 to about 15 mg/kg/per dose. In yet another embodiment, a Bcl-2 antisense oligonucleotide is administered locally at a dose of about 1 to about 50 mg/kg/per dose; preferably at a dose of about 4 to about 30 mg/kg/per dose; more preferably at a dose of about 5 to about 15 mg/kg/per dose. It will be evident to one skilled in the art that local administrations can result in lower total body doses. For example, local administration methods such as intra-tumoral administration, intraocular injection, or implantation, can produce locally high concentrations of Bcl-2 antisense oligonucleotide, but represent a relatively low dose with respect to total body. Thus, in such cases, local administration of a Bcl-2 antisense oligonucleotide is contemplated to result in a total body dose of about 0.1 to about 50 mg/kg/per dose.

In another embodiment, a particularly high dose of Bcl-2 antisense oligonucleotide, which ranges from about 10 to about 50 mg/kg/per dose, is provided during a treatment cycle.

Moreover, the effective dose of a particular Bcl-2 antisense oligonucleotide may depend on additional factors, including the type of cancer, the disease state or stage of disease, the oligonucleotide's toxicity, the oligonucleotide's rate of uptake by cancer cells, as well as the weight, age, and health of the individual to whom the antisense oligonucleotide is to be administered. Because of the many factors present in vivo that may interfere with the action or biological activity of a Bcl-2 antisense oligonucleotide, one of ordinary skill in the art can appreciate that an effective amount of a Bcl-2 antisense oligonucleotide may vary for each individual and can readily be discerned from the teachings herein the proper dose for a particular patient .

In another embodiment of the present invention, the Bcl-2 antisense oligonucleotide oblimerson is administered intermittently at a dose that will provide a mean peak plasma concentration (Cₘₐₓ) of at least 6 μg/mL, more preferably at least about 8 μg/mL, more preferably at least about 10 μg/mL, even more preferably at least about 15 μg/mL, more preferably at least about 20 μg/mL. In another embodiment the mean peak plasma concentration is at least about 40 μg/mL and more preferably at least about 50 μg/mL. Surprisingly, it has been found that when administered intermittently in accordance with the present invention, higher and more effective peak plasma levels can be achieved without clinically significant toxicity. The mean peak plasma concentration should be determined using plasma concentrations from samples of at least 4 patients and should be measured from 15 minutes up to 24 hours after initiation of the administration of a oblimersen treatment in order to determine to peak plasma concentration in each of the sample subjects. Plasma concentrations of oblimersen can be measured by anion exchange high-performance and thereby mean peak plasma concentrations. liquid chromatography. Briefly, duplicate standard curves can be produced in control plasma at the levels of oblimersen at 0.25, 0.5, 1, 2, 5, 10, and 20 mg/mL. Plasma from standard curves and patient samples are extracted with phenol:chloroform:isoamyl alcohol. Chromatographic separation is achieved on a GenPak-Fax column (Waters, Watford, United Kingdom). Oblimersen is eluted with a gradient of LiCl₂ 2 mol/L in Li(OH)₂ 20 mmol/L. Detection is achieved by spectroscopy at 254 nm. Oligonucleotide extraction can be performed by adding 0.5 mL of plasma to 2.45 ml of 0.4% sodium dodecyl sulfate, 50 mM NaCl, 10 mM EDTA, 10 mM Tris, pH 7.4, and vortex-mixed for 2 min. HPLC conditions: The HPLC system consists of two Kontron pumps, a gradient-former 460 and a Kontron autosampler. UV detection is performed at 254 nm with a Unicam diode-array detector. The HPLC column is a Waters GenPak-Fax column (4.6 3 100 mm), buffer A is 20% acetonitrile/10 mM Li(OH)₂ and buffer B is 20% acetonitrile/10 mM Li(OH)₂/2 M LiCl₂. A linear gradient is run from 10 to 100% buffer B over 30 min, with a flow rate of 0.5 ml/min. Eighty microliters of sample is injected into the autosampler. Fractions (0.5 ml) are collected with a Packard 1122 fraction collector, and 5 ml of Hionic Fluor scintillant is added to the samples, which is counted for 5 min. See Journal of Clinical Oncology, Vol 18, No 9 (May), 2000: pp 1812-1823 and the Journal of Pharmacology and Experimental Therapeutics, Vol. 281, No. 1, 420-427, 1997 which are hereby incorporated by reference.

Marcucci, et al., demonstrated that a steady state concentration (Css) was achieved within 24 h when a daily continuous IV infusion of Bcl-2 antisense oligonucleotide (Genasense®, G3139) at a dose of 4 mg/kg/d and 7 mg/kg/d were given to two cohorts of patients for 10 days but plasma concentrations decreased mono-exponentially and becomes undetectable within 4 hours. The plasma half life is about 60 minutes. (G. Marcucci, et al. (2003) Blood 101:425-432). When G3139 was delivered as a continuous subcutaneous infusion for 14 days, the steady state plasma levels was observed for 48 h after the beginning of infusion. The mean plasma half-life for elimination was 7.46 h. (See, Waters, et al. (2000) J. Clin. Oncol. 18:1812-1823). On the other hand, Yuen, et al., reported the plasma concentrations of a 20-length phosphorothioate oligonucleotide specific for PKC-A were similar at 24 h after the start of continuous IV infusion of the oligonucleotide and at day 21, i.e. the Css was achieved by 24 h. (See, A. R. Yuen, et al. (1999) Clin. Cancer Res. 5:3357-3363). The plasma half-life ranged from about 40 minutes after the 1.0 mg/kg/d to about 60 min after a 3.0 mg/kg/d dosing. These observations illustrate that the manner of administration is important in order to achieve a concentration in the plasma that is sufficient for transporting the oligonucleotide into the cell so as to be effective in inhibiting the Bcl-2 anti-apoptosis function.

The high dose may be achieved by administrations over a period, from minutes to hours, more preferably hours, as a continuous infusion on a single day of treatment or optionally as a bolus injection. A single administration of a high dose may result in circulating plasma levels of Bcl-2 antisense oligonucleotide that are transiently much higher than 30 μg/mL. Moreover, single administrations of particularly high doses of a Bcl-2 antisense oligonucleotide may result in a Bcl-2 antisense oligonucleotide Cₘₐₓ in much less than 12 hours. It is envisioned that in one embodiment of the invention, the high dose may be administered at intermittent intervals of about 2 to about 10 days, preferably at about 3 to about 9 days and more preferably at about 5 to about 7 days. In one preferred embodiment in a treatment cycle a single dose of a high concentration that is sufficient to achieve a Cₘₐₓ sufficient so as to reduce the amount of oligonucleotide necessary to treat a tumor is administered. In a further embodiment, large doses of the oligonucleotide may be administered by means of low doses given over long period on every other day.

Additionally, the dose of a Bcl-2 antisense oligonucleotide may vary according to the particular Bcl-2 antisense oligonucleotide used.

Other factors to be considered in determining an effective dose of a Bcl-2 antisense oligonucleotide include whether the oligonucleotide will be administered in combination with other therapeutics. In such cases, treatment with a high dose of Bcl-2 antisense oligonucleotide can provide a combination therapy wherein the amount of the other therapeutic is reduced resulting in a reduction in toxicity. For example, treatment of a patient with about 10, to about 50 mg/kg/dose of a Bcl-2 antisense oligonucleotide at intermittent intervals can further increase the sensitivity of a subject to cancer therapeutics. In such cases, the high dose of Bcl-2 antisense oligonucleotide administered in accordance with the present invention is combined with, for example, lower doses of a cancer therapeutic agent resulting in improved treatment of a patient in need thereof.

In one embodiment, an 18-base phosphorothioate Bcl-2 antisense oligonucleotide designated G3 139 (oblimersen; R. J. Klasa, et al. (2002) Antisense and Nucleic Acid Drug Development 12:193-213), which is complementary to the first six codons of the Bcl-2 mRNA and hybridizes to the respective target RNA bases, is administered at intermittent intervals of about 2 to about 10 days.

In another embodiment, oblimersen is administered at a dose of about 0.1 to about 10 mg/kg/administration. In a specific embodiment, oblimersen is administered at intermittent intervals of about 2 to about 10 days at a dose of about 1 to about 50 mg/kg/administration; more preferably at a dose of about 4 to about 30 mg/kg/administration, and most preferably at a dose of about 5 to about 15 mg/kg/administration. In another embodiment, oblimersen is administered at said dose at intermittent intervals of about 3 to about 9 days. In yet another embodiment, oblimersen is administered at said dose at intermittent intervals of about 4 to about 7 days. In a preferred embodiment, oblimersen is administered at said dose at intermittent intervals of about 3 to about 5 days. In a most preferred embodiment, oblimersen is administered at a dose of about 5 to about 30 mg/kg/dose at intermittent intervals of about 3 to about 14 days. The invention contemplates other preferred treatment regimens depending on the particular Bcl-2 antisense oligonucleotide to be used, or depending on the particular mode of administration, or depending on whether the Bcl-2 antisense oligonucleotide is administered as part of a combination therapy, e.g., in combination with a cancer therapeutic agent. The intermittent dose can be administered in one or more treatments.

Dosing and Scheduling Cycles

The present invention further provides methods and compositions for a dosing and scheduling cycle for administering an oligomer following administration of a pre-treatment discussed above. To date, studies using an oligomer, including Bcl-2 antisense oligonucleotide for treatment of cancer have treated patients by administering a daily dose of, for example, the Bcl-2 antisense oligonucleotide for a period of about 14 days or about 21 days followed by administration of a conventional therapy such as radiotherapy or chemotherapy with a rest period of about 7-day between the 14- or 21-day cycle. Although results from these studies have been encouraging, further improvement in the uptake of Bcl-2 antisense oligonucleotides was observed when mice bearing xenograft tumors were treated according to the present invention, receiving a pre-treatment of X-ray before being treated with intermittent high doses of Bcl-2 antisense oligonucleotides.

Thus, in one embodiment of the invention, it is contemplated that a cycle consists of administering to the patient a high dose of one or more oligomers after administration of a pre-treatment wherein the oligomer is dosed intermittently with about 2 to about 10 day intervals. A resting period of one to four weeks may follow before a second cycle is administered to the patient. A total of one to six cycles or more may be given to a patient.

Of course, it is to be understood and expected that variations in the principles of the invention herein disclosed can be made by one skilled in the art and it is intended that such modifications are to be included within the scope of the present invention.

All references mentioned herein are incorporated in their entirety.

### EXAMPLES

Oligonucleotides used in the experiments described below were all phosphorothioate oligonucleotides targeting bcl-2. The oligonucleotides used were HPLC purified and dissolved in sterile saline before use:

| | |
|---|---|
| G3139 | |
| 5′ - TCTCCCAGCGTGCGCCAT-3′ | anti-bcl-2 |
| | (bases +1 to +18) |
| FAM-G3139 | |
| F - TCTCCCAGCGTGCGCCAT-3′ | fluorescent G3139 |
| G3622 | |
| 5′ - TACCGCGTGCGACCCTCT-3′ | reverse polarity control |

### Example 1

### Higher Uptake of Fluorescent-Labeled Oligonucleotide, G3139 in Xenograft Tumor-Bearing Mice Treated Intermittently With High Dose Oligonucleotide

In vivo tumor model: PC-3-Bcl-2 xenograft tumors were established by injecting 1×10⁶ cells in 1 mg/mL MatrigelTM into the flanks of male athymic nude mice. When the average tumor size was 65 mm³, animals were randomized and treated via IP with fluoresceinate Bcl-2 antisense oligonucleotide (FAM-G3139) at a dose of 5 mg/kg daily for 7 days (low dose), or at a dose of 15 mg/kg (high dose) given intermittently on days 1, 4 and 7, i.e. at a 3-day interval between dosing. (FIG. 1A).

Uptake offluoresceinated G3139 oligonucleotide. On days 8 and 12, animals were sacrificed and subcutaneous and organs were resected. A portion of each resected tissue was either (a) embedded in OCT (Miles Laboratories) or (b) placed in cryotubes and snap-frozen (liquid N2) for cryosectioning (8-10 μM thickness), mounted on Superfrost-plus slides, (Fisher). Fluorescence intensity was recorded using a stereomicroscope (Zeiss, 40) and spot camera (QImaging).

The results in FIG. 1B show a higher uptake of fluoresceinated G3139 by the tumor cells of mice treated intermittently at days 1, 4 and 7 with 15 mg/kg oligonucleotide (high dose, middle panel) compared to mice treated daily with 5 mg/kg oligonucleotide (low dose, left panel) on day 8. On day 12, uptake of fluoresceinated G3139 was observed to be even higher in mice treated intermittently with 15 mg/kg G3139 (right panel).

### Example 2

### Pre-Pre-Treatment With X-ray Irradiation Enhanced Uptake of Fluorescent-Labeled Oligonucleotide, G3139 in Xenograft Tumor-Bearing Mice Treated Intermittently With High Dose Oligonucleotide

In vivo tumor model: PC-3-Bcl-2 xenograft tumors were established in the flanks of male athymic nude mice as described in Example 1 above. When the average tumor size was 65 mm³, animals were randomized and mice were either pre-treated with 5 Gy X-ray prior to administration of FAM-G3139 oligonucleotide by IP at a low dose of 5 mg/kg, daily for 7 days or at a high dose of 15 mg/kg on given intermittently on days 1, 4 and 7, or treated with X-ray on the last day after completing administration of G3139. (FIG. 2A).

Uptake offluoresceinated G3139 oligonucleotide. On day 11, animals were sacrificed and subcutaneous and organs were resected. The resected tissues were processed as in Example 1 described above and fluorescence intensity was recorded using a stereomicroscope (Zeiss, 40) and spot camera (QImaging).

FIG. 2B shows that pre-treatment with 5 Gy X-ray at the beginning of a cycle prior to administration of 5 mg/kg G3139 result in a higher uptake of fluoresceinated G3139 by the tumor cells than mice treated first with 5 mg/kg G3139 followed by X-ray at the end of the cycle.

FIG. 3 shows that uptake was further increased when the mice were pre-treated with 5 Gy X-ray at the beginning of a cycle when 15 mg/kg G3139 were administered at intermittent intervals.

### Example 3

### Pre-Pre-Treatment With X-ray Irradiation Prolonged Retention of Fluorescent-Labeled Oligonucleotide; G3139 in Xenograft Tumor-Bearing Mice Treated a Single Dose of G3139.

In vivo tumor model: PC-3-Bcl-2 xenograft tumors were established in the flanks of male athymic nude mice as described in Example 1 above. When the average tumor size was 65 mm³, animals were randomized and the right flank of the xenograft tumor bearing mice were pre-treated with 5 Gy X-ray prior to administration of fluoresceinated G3139 oligonucleotide intravenously at a low dose of 6 mg/kg, daily for 5 days (top left panel); at a high dose of 10 mg/kg on given intermittently on days 1, 3, and 5 (top middle panel); a single dose of 30 mg/kg on the same day of X-ray pre-treatment (top right panel); and non-treated left flank bearing xenograft tumor (bottom 3 panels). Each mouse received a total dose of 30 mg/kg fluorescent G3139.

Uptake offluoresceinated G3139 oligonucleotide. On day 8, animals were sacrificed, subcutaneous and organs were resected, and counterstained with DAPI as an internal staining control for nucleus staining. The resected tissues were processed as in Example 1 described above and fluorescence intensity was recorded using a stereomicroscope (Zeiss, 40) and spot camera (Qimaging). The signal intensity was recorded in three areas of each sample and quantified using MCID Elite (Imaging Research) and averaged to yield the reported intensities.

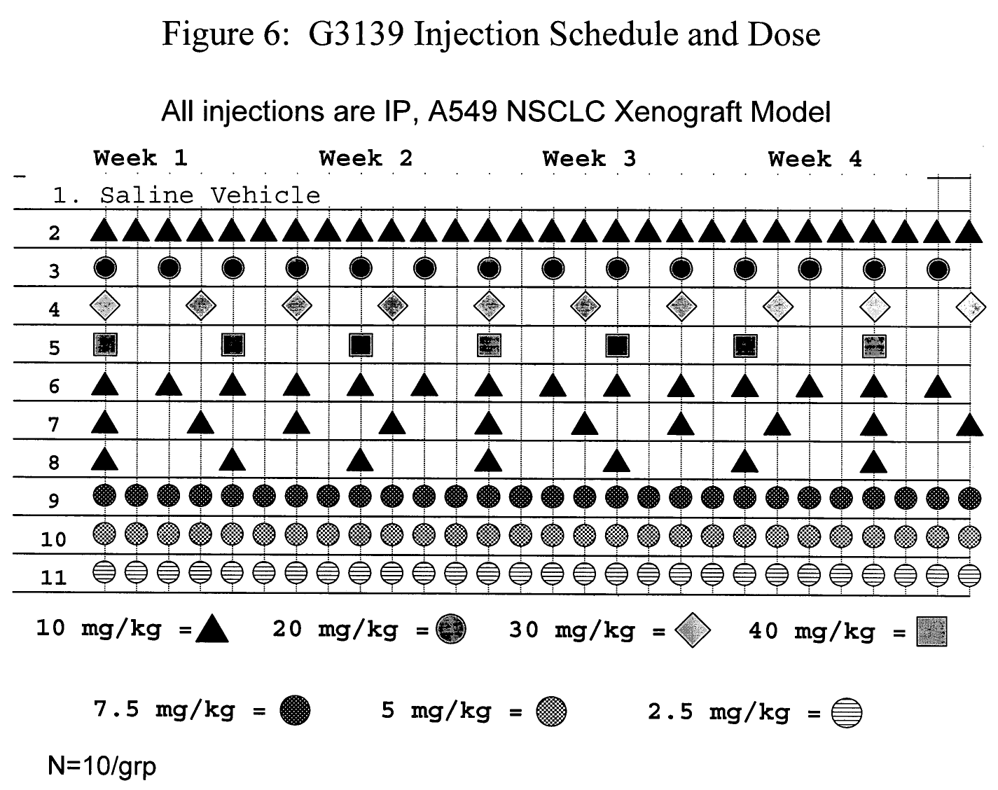

FIG. 6 shows that pre-treatment with X-ray on day 1 followed by a single 30 mg/kg dose of G3139 (top right panel) demonstrated not only enhanced uptake of the oligonucleotide but persistence 7 days after administration of both radiation and G3139. Uptake of fluorescent G3139 is higher in mice treated with intermittent doses of 10 mg/kg G3139 (top middle panel) than mice treated with a daily dose of 6 mg/kg for 5 days (top left panel). There is no visible staining of tumor cells from the left flank that had not been sensitized with X-ray.

FIG. 5 shows the quantitation of FAM-3139 in the tumors from the three groups of animals. The graph shows that uptake is highest in tissues from mice treated first with X-ray followed by a single dose of 30 mg/kg FAM-3139 than either mice treated with intermittent doses of 10 mg/kg (every other day, total of 3 doses) or mice treated with daily dose of 6 mg/kg for 5 day.

### Example 4

### Intermittent Dosing of Bcl-2 Antisense Increase Antineoplastic Activity as a Single Agent

In vivo tumor model: NSCLC xenograft tumors were established in the flank of male athymic nude mice by injecting subcutaneously 5 to 9×10⁶ A549 tumor cells. When the average tumor size was 65 mm³, animals were randomized and mice were either injected intraperitoneally (IP) with G3139 at the following doses of 2.5, 5, 7.5 mg/kg daily for 4 weeks (28 days), 10 mg/kg daily for 4 weeks (28 days), every other days for 4 weeks (total 14 days), every two-days for 4 weeks (total 10 days), and every three-days for 4 weeks (total 7 days). Twenty mg/kg injections every other days for 4 weeks (total 14 days) were also carried. At 30 mg/kg and 40 mg/kg, mice were injected every two- or three-day intervals respectively. (FIG. 6). The tumor growth were measured 15 - 55 days post-implantation and the tumor volumes were calculated based on the formula: [(width)²×length]/2. FIG. 7 shows that the tumors in mice treated with 20 mg/kg of G3 139 every other day grew slower than mice injected daily with 10 mg/kg (total 280 mg/kg). Tumors in mice injected with higher doses but interminttently with 2-3 days intervals also grew slower than mice treated with daily doses at lower doses.

Survival of xenograft tumor bearing mice was monitored for 75 days post implantation. Per cent survival of the mice injected with various doses using schedules depicted above is shown in FIG. 8.

We claim:

1. A method for treating a human patient with a neoplastic disease or an autoimmune disease in need of treatment thereof, comprising administering at least one treatment cycle to the patient wherein said treatment cycle comprises:
i. an effective amount of a pretreatment, wherein the pretreatment is selected from the group consisting of one of more of radiotherapy, acoustic therapy, thermal therapy, and anti-neoplastic therapy; and
ii. an effective dose of an oligomer providing a mean peak plasma concentration of oligomer from about 6 μg/mL to about 50 μg/mL.

2. The method of claim 1, wherein the oligomer is selected from the group consisting of oblimersen, G4460, and INX-3001.

3. The method of claim 1, wherein the mean peak plasma concentration is at least about 40 μg/mL.

4. The method of claim 1, wherein the mean peak plasma concentration is at least about 20 μg/mL.

5. The method of claim 1, wherein the mean peak plasma concentration is at least 15 μg/mL.

6. The method of claim 1, wherein the anti-neoplastic therapy is selected from the group consisting of one or more of pharmacologic therapy, and biological therapy.

7. The method of claim 1, wherein the oligomer is administered at a frequency selected from every second day, every third day, or every fourth day.

8. The method of claim 1, wherein in the treatment cycle, the pretreatment is radiotherapy administered one day preceding administration of a single dose of the oligomer.

9. The method of claim 8, wherein in the treatment cycle, the pretreatment comprises radiotherapy and a single dose of about 30 mg/kg of the oligomer administered on the same day.

10. The method of claim 1, wherein in the treatment cycle the pretreatment comprises administration of radiotherapy one day preceding administration of a initial dose of about 4 mg/kg to about 30 mg/kg of the oligomer, and administration of at least one subsequent dose of the oligomer wherein the first subsequent dose is administered from about one to about five days after the initial dose.

11. The method of claim 10, wherein the subsequent doses of the oligomer comprise about 6 mg/kg to about 10 mg/kg of oligomer administered every other day.

12. The method of claim 11, wherein the subsequent doses of the oligomer comprise 6 mg/kg/dose administered daily for five days.

13. The method of claim 10, wherein the subsequent doses of the oligomer are about 15 mg/kg per dose.

14. The method of claim 13, wherein the subsequent dose of the oligomer is about two days after the initial dose.

15. The method of claim 10, wherein the oligomer is oblimersen.

16. A pharmaceutical composition for treating a human patient with a neoplastic disease or an autoimmune disease who is pre-treated with radiotherapy in a treatment cycle, said pharmaceutical composition being suitable for administering to the patient in each cycle of treatment an effective dose of an oblimersen providing a mean peak plasma concentration of oligomer from about 6 μg/mL to about 50 μg/mL.
